(19) United States
(12) Patent Application Publication
(10) Pub. No.: US 2009/0326975 A1
(43) Pub. Date: Dec. 31, 2009

(54) SYSTEMS AND METHODS FOR CONTROLLING A REPLENISHMENT PROGRAM THROUGH A CONTRACT PHARMACY

(75) Inventors: Jason Michael Hardaway, Portland, OR (US); Daniel Dean Sawyer, Portland, OR (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: WELLPARTNER INCORPORATED, Portland, OR (US)

(21) Appl. No.: 12/145,960

(22) Filed: Jun. 25, 2008

(51) Int. Cl. G06Q 50/00; G06Q 10/00

(52) U.S. Cl. 705/2

(57) ABSTRACT

The existing infrastructure of a pharmacy may be extended to allow the pharmacy to service patients of one or more qualified entities that are entitled to discounted drugs through one or more qualified programs (e.g., 340B, GPO, PAP, or the like). One or more data structures of the systems already in place at the pharmacy may be leveraged to store data relating to the qualified entity and/or qualified program that covering the patients served by the pharmacy. This data used to comply with the regulatory requirements of the qualifying programs under which the drugs are obtained. The data may also be used to track inventory loaned to the qualifying entity from a common inventory of the pharmacy. The inventory information may be used to replenish the loaned inventory as needed, identify alternative, equivalent drugs for use in replenishment, and manage the reimbursement and financial reconciliation elements of the qualified programs.

### TECHNICAL FIELD

This disclosure relates generally to techniques for allowing a contract pharmacy to serve patients of a qualified entity (patients may include persons qualifying for coverage under according to the regulations governing the qualified entity, such as certain qualifying persons, employees, and/or their dependents) that are entitled to discounted drugs under a qualified program.

### BRIEF DESCRIPTION OF THE DRAWINGS

Non-limiting and non-exhaustive embodiments of the disclosure are described, including various embodiments of the disclosure with reference to the figures, in which:

FIG. 1A is a block diagram of a system for distributing prescribed drugs to patients of a qualified entity entitled to discounted drugs under a qualified program.

FIG. 1B is a block diagram of a qualified program management module.

FIG. 2 is a flow diagram of one embodiment of a method for allowing a contract pharmacy to service patients of a qualified entity entitled to discounted drugs under a qualified program.

### DETAILED DESCRIPTION OF PREFERRED EMBODIMENTS

There are a number of drug discount programs available to consumers. Some of these programs include the federal 340B program (Section 602 of Public Law 102-585, the Veteran's Health Care Act of 1992, enacted Section 340B of the Public Health Services Act), also known as section 602 or “PHS” pricing. The 340B program may provide access to low-cost drugs for the patients of certain qualifying entities, including Community Health Clinics, Disproportionate Share Hospitals, and other safety-net organizations. The 340B program is not a governmental purchasing program, but is a discount program which is overseen by the federal government, Office of Pharmacy Affairs. Eligibility for participation in a 340B program is determined by the entities' status, specifically by receiving one of several grants or by being a certain type of disproportionate-share hospital or federally qualified health center (FQHC) or look-alike. Eligible health care entities include safety-net entities and a number of health facilities. Various FQHCs may be eligible health care entities, such as consolidated health centers, migrant health centers, health care for the homeless, school-based health centers, public housing health centers, PL 93-638 tribal health centers, urban Indian health centers, and qualified community health clinics. Other eligible entities may also include FQHC look-alikes, native Hawaiian health centers, Ryan White Care Act Grantees, Title X Family Planning, black lung clinics, comprehensive hemophilia diagnostic treatment centers, state or locally funded centers treating sexually transmitted diseases or tuberculosis, certain disproportionate share hospitals, and other safety-net organizations. Pharmaceutical manufacturers, as a condition of participation in state Medicaid programs, are required to sell covered drugs to such qualified entities (e.g., entities that qualify for coverage under 340B) at or below a statutorily defined “ceiling price”.

Other organizations may take advantage of their status as non-profit institutions to reduce pharmacy expenditures for their employees, retirees, dependents, and other qualified individuals by participating in a purchasing cooperative known as a Group Purchasing Organization (GPO). A GPO may save significantly on pharmaceutical drugs purchased for their members' “own use.” Other programs, such as Patient Assistance Programs (PAP) may also provide significant drug discounts. Finally, for-profit organizations that cannot participate in any of the previous programs may wish to directly purchase medications for their employees and dependents and distribute them through contract pharmacies.

The programs mentioned above may be referred to generally as a “qualified program” and an entity eligible to participate in such a program may be referred to as a “qualified entity.” Moreover, although the 340B, GPO, and PAP discount programs are specifically mentioned; as used herein, these programs are used as examples of qualified programs. The teachings of this disclosure may extend to any discount program and related regulatory scheme. As such, this disclosure should not be read as limited to any particular set of qualified programs and/or any particular set of qualified entities eligible to participate in such programs.

Most of the qualified programs discussed above (340B, GPO, and PAP) are heavily regulated. In some cases, failure to follow the regulations of a particular qualified program may result in regulatory enforcement action and/or result in criminal changes. For example, failure to follow the regulatory requirements associated with a qualified program may result in termination from participation in the program (e.g., a pharmacy and/or entity operating in violation of the regulations may be disbarred from participation in the qualified program).

Due to the complexity of regulations for many qualified programs, many eligible entities and general service contract pharmacies are unable to support these programs. This is due in part to the large overhead required to comply with the regulatory requirements of the programs and/or the complex inventory management and financial reconciliation inherent in such qualified programs. Such regulations may relate to reporting, inventory management, financial reconciliation, and/or replenishment of inventory.

One way that contract pharmacies may be able to service the patients of qualified entities (e.g., entities who qualify for one or more qualified programs entitling them to low-cost drugs) is by adapting the adjudication and/or Coordination of Benefits (COB) feature systems already in place at many pharmacies. Traditionally, these systems have been used to manage benefits. Many adjudication systems include COB systems, which have been employed where a patient is covered by two different insurance carriers. For example, a child may be covered by both his mother's health insurance and his father's health insurance. As such, when the child presents a prescription to a pharmacy, the two health insurance payers may be accountable for reimbursement of the related claim(s) (at various levels). In this case, a first adjudication (e.g., determine the co-pay or other cost to be borne by the patient, payer 104 and/or the qualified entity 102 covering the patient 104) of the prescription claim may be made to a first payer (e.g., a first insurer), and a second adjudication of the prescription may be made to a second payer (e.g., a second insurer). The adjudication system is extended and the process adapted to implement program compliance and provide information necessary to support inventory management, financial reporting, and program reporting. Since adjudication programs are in wide use among contract pharmacies, it may be desirable to adapt a variation of the adjudication system for use in fulfilling the regulatory requirements of qualified programs (e.g., to tracking inventory dispensed to patients of qualified entities). Many adjudication systems use a common data format. An example of one such data format is the National Council for Prescription Drug Programs (NCPDP) 5.1 specification. By extending the use of these data structures and systems to include information relating to the qualified entity and/or qualified plan used in the adjudication, the adjudication data structures (e.g., the NCPDP 5.1 compliant data structures) may be utilized to provide inventory management (e.g., allowing the contract pharmacy to “loan” medication inventory to the qualified entity) and replenishment pharmacy functions to support one or more qualified programs. A pharmacy may leverage its existing adjudication systems and/or infrastructure to support the highly-complex inventory and financial management components of these qualified programs. Although the NCPDP 5.1 is specifically mentioned herein, there are many variations of the adjudication system and data structures in the art, any of which may be modified and/or adapted for use in a replenishment pharmacy system. As such, this disclosure should not be read as limited to any particular adjudication data structure, inventory loan structure, or purchasing program.

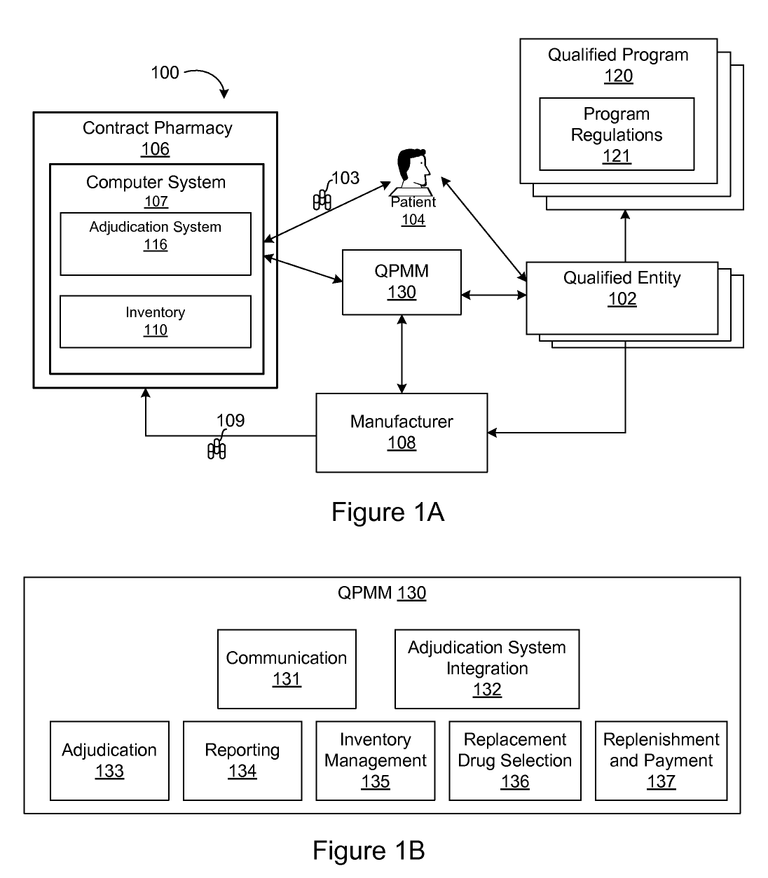

FIG. 1A is a block diagram of one embodiment of a system 100 for providing replenishment pharmacy functionality in a contract pharmacy 106. A plurality of qualified entities 102 provide health care services and/or health insurance services to qualified patients 104. Drugs are dispensed to the patients 104 through a contract pharmacy 106 under the supervision of a qualified entity 102, such as a health care provider (e.g., a hospital), non-profit organization, insurer, or the like.

The contract pharmacy 106 may provide drugs to the eligible patients of the qualified entities 102 as a loan from its current inventory. The eligible qualified entity 102 may purchase discounted replenishment drug inventories from pharmaceutical manufacturers 108 for shipment to the contract pharmacies that dispense such drugs on behalf of the eligible qualified entities 102. As can be appreciated, a wide variety of qualified programs 120 may be involved in providing health care services and dispersing drugs; however, these programs and the manner in which they are managed impact both pricing and access to drugs.

The qualified entities 102 may participate in one or more qualified programs 120 that allow one or more of the qualified entities 102 to obtain low-cost prescription drugs. As discussed above, the eligibility of the qualified entities 102 to participate in one or more qualified programs 120 (e.g., 340B, GPO, PAP, or the like) may be established by federal statute and be covered by respective program regulations 121.

Patients who receive drugs through a qualified program 120 must be a qualifying patient 104 of a qualified entity 102 (e.g., be eligible for coverage from the qualified entity 102 according to its respective qualified program 120). A deviation of this requirement would result in the diversion of drugs through the qualified program 120. Diversion is the distribution of drugs purchased under a qualified program 120 to a non-eligible patient, either intentionally or unintentionally. The program regulations 121 defining most qualified programs 120 prohibit all forms of medical resale or diversion.

The program regulations 121 governing the qualified programs 120 may also prohibit “double-dipping.” Double dipping occurs when qualified entities 102 receive drugs under an qualified program 120 for which a State Medicaid agency (as the payer) will also request a rebate under an OBRA 1990 rebate mechanism related to the claim associated with the drug dispensed through the qualified program 120. With this prohibition, a qualified entity 102 can receive a discount through the qualified program 120, or a State Medicaid agency can receive a discount via rebate; however, both discounts may not occur for the same drug claim.

A qualified entity 102 may provide its patients 104 access to discounted drugs 103 through a contract pharmacy 106 (e.g., a contract pharmacy having a contractual arrangement with one or more of the qualified entities 102 to distribute drugs to the member patients 104 of the qualified entities 102). As such, the contract pharmacy 106 may comprise a standard, outpatient pharmacy capable of servicing patients that are either covered by qualified entities 102, are cash-pay customers, and/or are covered by other, non-qualified entities.

Under a qualified program 120, a qualified entity 102 may purchase or can make available, program-specific discounted drugs to replenish drug inventory dispensed by the contract pharmacy 106 to the patients 104 of the qualified entity 102. In order to properly replenish such medication, a Qualified Program Management Module (QPMM) 130 may be used to track the drug dispensed to the patients of the qualified entity 102. Using the QPMM 130, the contract pharmacy 106 may dispense drugs to the patients 104 of the qualified entity 102 from its inventory 110 (e.g., the contract pharmacy 106 may not need to maintain separate inventories of “qualified entity” drugs). By so doing, the contract pharmacy 106 essentially “loans” a portion of its inventory 106 to the qualified entity 102. The qualified entity 102 may then replenish the inventory 110 of the pharmacy with discounted drugs obtained according to the program regulations 121 of its respective qualified program 120.

A “bill-to/ship-to” arraignment is one way in which the qualified entity 102 may replenish inventory 110 distributed to its patients 104 via the contract pharmacy 106

Under a “bill-to/ship-to” arrangement, replenishment drugs 109 are shipped by a drug provider, such as a drug manufacturer 108, wholesaler, or the like directly to the contract pharmacy 106 that dispensed the drugs 103 to the patients 104 of the qualified entity 102 (as a replenishment of such loaned inventory), while the drugs 109 are purchased by the qualified entity 102. Although FIG. 1 shows a drug manufacturer 108, the teachings of this disclosure may be used with any drug and/or medication provider known in the art including, but not limited to: a drug maker, a distributor, a wholesaler, a reseller, another retailer and/or contract pharmacy, or the like. Various qualified programs 120 may comprise different replenishment models according to their respective program regulations 121.

As discussed above, the program regulations 121 of the respective qualified programs 120 may be very complex and may include detailed regulations relating to claim adjudication, reporting, inventory management, replacement drug selection, and payment management for the qualified programs 120. The program regulations 121 of various qualified plans 120 may be significantly different. Moreover, the program regulations 121 defining the qualified plans 120 may change over time (e.g., with changes to the statutes and/or rules defining the qualified programs 120). As such, managing the regulatory schemes of the qualified programs 120 may not be practical or even possible for a qualified entity 102, which desires to utilize a contract pharmacy 106 for the delivery of discounted drugs to eligible patients.

As discussed above, the adjudication system 116 of the contract pharmacy 106 may be leveraged to provide a additional functionality (e.g., the adjudication system 116 may be put to uses other than standard adjudication processes, such as Coordination of Benefits (COB) processing). The adjudication system 116 and/or inventory 110 system of the contract pharmacy may be implemented as a computer system 107. The computer system 107 may be distributed throughout various contract pharmacy 106 locations (e.g., the contract pharmacy 106 may have numerous storefront locations from which drugs may be distributed).

A Qualified Program Management Module (QPMM) 130 may leverage the adjudication system's 116 functionality to thereby allow the contract pharmacy 106 to serve patients 104 of one or more qualified entities 102 according to the program regulations 121 of their respective qualified programs 120. In this way, the contract pharmacy's 106 adjudication system 116 may be leveraged for additional uses beyond supporting industry standard adjudication and/or COB functionality.

As described above, the adjudication system 116 may run on one or more computer systems 107 of the contract pharmacy 106. As is well known in the art, the computer systems 107 may comprise a processor (not shown), an output device (not shown) in electrical communication with the processor, and a memory (not shown) electrically coupled to the processor and output device. The memory (not shown) may comprise an operating system and a plurality of applications. The adjudication system 116, the QPMM 130, and/or an inventory management system 110 may be implemented as one or more applications running in coordination with and capturing required adjudication data from the contract pharmacy's 106 computer systems 107. Alternatively, or in addition, the QPMM 130 may be implemented on a separate computer system from the computer systems 107 of the contract pharmacy 106 (e.g., as a stand-alone service or the like). In such embodiments, the QPMM 130 may be communicatively coupled to the computer system 107 of the contract pharmacy 106 via a communications network (not shown), such as a Local Area Network (LAN), Wide Area Network (WAN), Virtual Private Network (VPN), the Internet, or the like.

In some embodiments, the QPMM 130 may extend the data structures used by the adjudication system 116. This may comprise inserting data relating to a particular qualified program 120 or the like into an adjudication system 116 data structure, such as a NCPDP 5.1 data structure. The QPMM 130 may use the qualified program-related information to adjudicate the prescription transaction (e.g., determine the co-pay or other cost to be borne by the patient 104 and/or the qualified entity 102 covering the patient 104), to generate reporting information required by the qualified program 120, to perform inventory management, determine appropriate replenishment medication, and manage payment for the replenishment medication in accordance with program regulations 121 of the qualified program 120.

As discussed above, some qualified programs 120 (e.g., 340B programs) interact with the contract pharmacy 106 using a replenishment model. The replenishment model enables contract pharmacies 106 to manage the inventory for qualified programs 120 virtually by managing the replacement of dispensed drugs on a replenishment basis. This precludes the need for the pharmacy to maintain separate physical inventories for each of the qualified entity 102 it services and/or its standard patients (e.g., those patients not served or covered by a qualified entity 102).

In one implementation, the replenishment model provides a form of inventory control. The replenishment model allows a contract pharmacy 106 to dispense drugs 103 to the patients 104 of covered and/or serviced by qualified entities 102 from its own (non-eligible program) common inventory 110, and then have that inventory replenished by the qualified entity 102. As described above, the contract pharmacy 106 effectively “loans” the qualified entity 102 the drug, and the qualified entity 102 then orders replacement inventory to “pay back” the contract pharmacy 106 for the loan.

The advantage of this so-called replenishment approach is that it reduces the likelihood of drug diversion since there is no inventory specific to any particular qualified program 120 sitting on the contract pharmacy's 106 shelves. The virtual nature of the inventory (as managed by the inventory management system 110) may be particularly important where the contract pharmacy 106 serves patients 104 from multiple qualified entities 102 under multiple qualified programs 120, since attempting to manage physically separate inventories in this case would quickly become untenable.

The replenishment approach described above may help the contract pharmacy 106 comply with the program regulations 121 of the qualified programs 120. For example, if the contract pharmacy 106 were to attempt to maintain separate physical inventories (not shown) for each qualified entity 102, there would be a chance that the pharmacy 106 could make errors in dispensing from the wrong inventory in connection with the wrong qualified entity 102. As discussed above, the inventory purchased under a particular qualified program 120 must only be used for eligible patients of that qualified program 120 or the result is diversion. This could cause the contract pharmacy 106 to run afoul of the qualified program 120 (e.g., diverting and/or double-dipping) resulting in disbarment of the contract pharmacy 106 (e.g., removing the ability for the contract pharmacy 106 to service patients covered by the qualified entity 102 and/or qualified program 120) or even criminal charges.

Of course, the replenishment model may be complicated by the fact that various qualified programs 120 may incorporate different regulatory schemes and/or program regulations 121 regarding how drugs are replenished to the contract pharmacy 106. For example, under a 340B program, replenishment drugs 109 may be purchased by the qualified entity 102 and shipped directly from the manufacturer 108 to the contract pharmacy 106. The QPMM 130 may be configured to support this so-called “bill-to/ship-to” arrangement, wherein replenishment drugs 109 may be ordered directly from the manufacturer 108 for shipment to the contract pharmacy 106 with payment by the qualified entity 102. However, other qualified programs 120 may not allow for this type of transaction. As such, the QPMM 130 may be configured to perform the appropriate replenishment processes according the program regulations 121 of a particular qualified program 120.

The inventory management and replenishment functions of the QPMM 130 may provide the additional benefit of reducing costs for the qualified entity 102, contract pharmacy 106, and/or patient 104. Generally, in the replenishment model, after the qualified entity 102 provides the contract pharmacy 106 with replacement drugs, the qualified entity 102 recoups from the contract pharmacy 106 any revenues generated for the drug minus dispensing and other service fees payable to the contract pharmacy 106. Accordingly, the larger the differential between the reimbursement amount of the dispensed drugs and the replacement cost thereof, the greater the savings for the qualified entity 102.

Some manufacturers 108 (or other drug providers, such as wholesalers, distributors, or the like) may discount drugs at certain times (e.g., at the end of the quarter, end of the month, etc.) and/or may occasionally offer certain drugs at steeply discounted promotional prices. The replenishment functionality of the QPMM 130 may be configured to take advantage of such discounts when they are available and when permitted by the program regulations 121 of the qualified program 120. Since the QPMM 130 is part or and/or communicatively coupled to the computer system 107 of the contract pharmacy 106 (e.g., coupled to the adjudication system 116 of the pharmacy 106), the QPMM 130 may monitor the national drug code (NDC) of the drugs dispensed to eligible patients 104 for the qualified entities 102. Using the NDC, and as part of its inventory management function, the QPMM 130 may determine one or more alternative, equivalent drugs to use in replacing the drugs dispensed to the patient 104 of the qualified entity 102. An alternative, equivalent drug may have a different NDC number than the drug dispensed to the eligible patient 104, but may be chemically and biologically equivalent thereto (e.g., the same drug produced by a different manufacturer, distributed by a different wholesaler, or the like). The QPMM 130 may determine whether there are pricing advantages to replenishing the contract pharmacy 106 with an equivalent drug and/or whether the qualified program 120 is able to purchase the equivalent drug. If so, the QPMM 130 may present the option to the contract pharmacy 106 at the time the drug is to be dispensed. In this way, the QPMM 130 may reduce costs of the pharmacy 106 and qualified entity 102 through NDC selection based on analysis of historic pricing discounts and communicating that information to the pharmacy prior to the dispensing of a drug (to an eligible patient) to promote the use of the most cost effective drug in connection with that eligible program.

FIG. 1B is a block diagram of one embodiment of a QPMM 130. The QPMM 130 may comprise communications, analytics, and data storage modules 131, which may allow the QPMM 130 to communicate with systems (e.g., computing devices and the like) of a pharmacy (not shown), a qualified entity (not shown), or the like. The communications module 131 may be configured to interact with an adjudication system of the pharmacy (not shown). As discussed above, in some embodiments, the QPMM 130 may be implemented as a separate computer system from the computer systems of the contract pharmacy (e.g., as a standalone service). As such, the communications, analytics, and data storage modules 131 may be configured to communication with the computer systems of the contract pharmacy over a network, such as a LAN, WAN, VPN, Internet, or the like.

The adjudication integration component 132 may be configured to extend the functionality of an existing adjudication system in the pharmacy. As discussed above, the adjudication integration component 132 may extend and/or override one or more fields in one or more adjudication system data structures to include information relating to a qualified entity and/or qualified program so that information may be captured by the QPMM 130. The modified adjudication system data structures may then be used by the other components of the QPMM 130 to manage the information relating to qualified program requirements, including: adjudication 133, reporting 134, inventory management 135, replacement drug selection 136, and/or inventory replenishment and financial reconciliation 137 modules.

The QPMM 130 may comprise a claim adjudication module 133, which injects qualified program and/or qualified entity information into a data structure of the adjudication system during the contract pharmacy's adjudication process (e.g., as drug is dispensed to a patient). In addition, the adjudication module 133 may interact with the replacement drug selection module 136 to determine whether an alternative, equivalent drug (e.g., a drug having a different NDC than the dispensed drug) is available to replenish the drug dispensed to the patient at a reduced cost.

The reporting module 134 processes qualified entity/qualified program data in the adjudication system's data structures and generates reporting information per the requirements of the qualified programs and/or qualified entities served by the QPMM 130. As discussed above, qualified programs, such as 340B, GPO, and/or PAP programs may impose significant reporting requirements. These reporting requirements may be used to prevent regulatory violations (e.g., prohibitions against diversion and/or double dipping) or other abuses of the qualified program. The reporting module 134 may be configurable so that its operation may be modified responsive to changes in the requirements of the regulatory schemes governing qualified entities and/or the creation of new qualified programs and/or qualified entities.

The inventory management module 135 may be configured to receive the data generated by the reporting module 134 to thereby track the drugs “loaned” to each particular qualified entity by the contract pharmacy. This data may later be used to drive the replacement selection module 136 and/or the replenishment and payment module 137.

The replacement drug selection module 136 may be used to identify replenishment medication for use in replacing contract pharmacy inventory dispensed to the patients of one or more qualified entities. As discussed above, in some cases a qualified entity may be eligible to purchase medication at reduced cost. In addition, drug manufacturers and/or distributors (e.g., wholesalers) may occasionally discount certain medications. The qualified entity may be entitled to purchase such discounted medications. The replacement drug selection module 136 may be configured to receive an NDC of the drug(s) to be replenished to the contract pharmacy, and to determine one or more alternative, equivalent drugs (drugs having different NDCs) for use in replacement. In some embodiments, the replacement drug selection module 136 may present the alternative, equivalent drugs to the contract pharmacy and/or qualified entity for approval. The alternative equivalent drugs may be used in place of the dispensed drugs upon the approval of the contract pharmacy and/or qualified entity. Alternatively, the replacement drug selection module 136 may be configured to automatically select desirable alternative, equivalent drugs.

The replenishment and payment module 137 may be used to replenish the medication to the contract pharmacy according to the inventory identified by the inventory management module 135 using the drugs identified by the replacement drug selection module 136. The replenishment and payment module 137 may be configured to replenish the drugs according to the requirements (e.g., regulations) of the qualified program of the particular qualified entity. As discussed above, qualified programs may have different regulations pertaining to how drugs are purchased for plan members. For example, some qualified programs allow replenishment using a “bill-to/ship-to” replenishment model, while others do not). Accordingly, the replenishment and payment module 137 may be configured to replenish medication according to the appropriate qualified program(s). Moreover, the replenishment and payment module 137 may be configured to allow for changes to the requirements of various qualified programs and/or the creation of new qualified programs. In addition, the replenishment and payment module 137 may facilitate the transfer of payment from the qualified entity to the drug manufacturer and/or distributor and from the pharmacy to the qualified entity. Again, the payment transfer may be performed according to the regulatory requirements of the qualified program associated with the qualified entity.

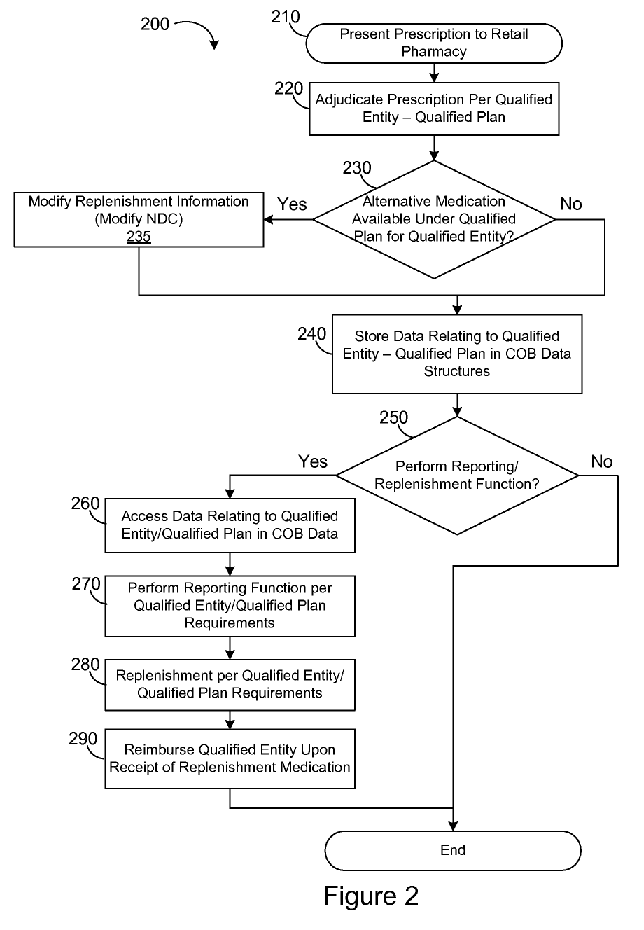

FIG. 2 depicts one embodiment of a flow diagram of a method 200 for controlling a replenishment program in a contract pharmacy. At step 210, a patient served and/or covered by a qualified entity may present a prescription to a contract pharmacy. At step 220, the pharmacy may adjudicate the prescription claim. The adjudication of step 220 may comprise providing the drug to the patient, determining the patient's portion of the cost of the drug (e.g., the co-pay), or the like.

During the claim adjudication step 220, information identifying the patient's qualified entity and/or qualified program may be obtained. At step 230, this information relating to the adjudication is gathered and stored in a data structure of the contract pharmacy's adjudication system. Such information may relate to the qualified entity that provides coverage and/or care to the patient, and/or the qualified program used by the qualified entity to obtain discounted drugs.

At step 230, the process 200 may determine whether a lower cost alternative drug is available to replace the drug dispensed to the patient at step 220. As discussed above, in some cases, an alternative, chemically and biologically equivalent drug may be available from an alternative source (e.g., manufacturer and/or distributor). Available alternative, equivalent drugs may be identified using the NDC of the drug dispensed at step 220. The nature of the qualified entity and/or the qualified program may determine whether a particular discounted drug is available. If an acceptable low-cost alternative is found, the flow may continue to step 235, wherein the alternative NDC may be recorded; otherwise, the flow may continue to step 240.

At step 240, the data relating to the qualified entity, qualified program, patient, and prescribed drug is passed to the QPMM system where it is processed and stored. This may comprise transmitting the information to an external computer processing system over a network (e.g., LAN, WAN, VPN, Internet, or the like). The identified methodology supports unambiguous processing and identification of qualified claims. In this way, the existing pharmacy infrastructure may be extended to support the patients of qualified entities and/or qualified programs using the QPMM system. The QPMM system in turn supports the communication, reporting, inventory management, replenishment, and business processing necessary to provide a complete business solution and meet the strict regulations required to implement the replenishment program mandated by the requirements of the qualified entities and respective qualified programs.

At step 250, method 200 may determine whether reporting, replenishment, or business management is required. The nature and timing of the reporting may vary depending on the particular qualified program and/or qualified entity serviced by method 200. Similarly, the determination may be dictated by the regulations of the qualified program and/or qualified entity. For example, replenishment may be required after a certain quantity of drugs have been dispensed to the patients of the qualified entity by the contract pharmacy (e.g., “loaned” to the qualified entity), and/or the inventory loaned to the qualified entity by the pharmacy has been depleted to a threshold level. If reporting and/or replenishment is required, the flow may continue to step 260; otherwise, the flow may terminate until such time that reporting and/or replenishment is required.

At step 260, the data gathered during the contract pharmacy adjudication process and stored in a data structure of the contract pharmacy's adjudication system may be accessed and filtered to extract any data relating to a particular qualified program and/or qualified entity used or otherwise identified in the adjudication process. At step 270, the filtered data may be used to generate one or more reports for the pharmacy, qualified entity, and/or qualified program. The reporting data may indicate the amount and nature of drugs distributed to the patients of a particular qualified entity under a particular qualified program, any replenishment actions taken, and the like. In alternative embodiments, the access and filtering of step 260 may be performed on a continual basis (e.g., as prescriptions are adjudicated for patients of the particular qualified entity). As discussed above, in some embodiments, the systems for performing the adjudication, reporting, inventory management, drug selection, and/or replenishment and payment services described herein may be separate from the computer systems of the contract pharmacy. As such, step 270 may comprise transmitting adjudication data (e.g., adjudication system data structures) to an external computer system via a network or other communication means.

At step 280, method 200 may replenish the drug according to the regulations of the particular qualified program and/or qualified entity identified in the data structures of the adjudication system. If a “bill-to/ship-to” arraignment is allowed by the qualified program and/or qualified entity, the method 200 may dispatch an order for replenishment drugs to a manufacturer and/or wholesaler to be shipped to the pharmacy. The billing for the order may be sent directly to the qualified entity. As discussed above, in some cases the drugs may be replenished from a different manufacturer and/or wholesaler (e.g., have a different NDC number) than that from which the drug dispensed at step 220 was originally sourced.

At step 290, the replenishment drugs may be received and the pharmacy may reimburse the qualified provider with the difference between the disbursement price of the drug and the replenishment cost paid (minus any overhead and service fees charged by the pharmacy). Of course, the reimbursement step of 290 may be omitted if the replenishment method used at step 280 does not require reimbursement and/or reimbursement is prohibited by the qualified program and/or qualified entity.

The described features, structures, or characteristics discussed in this disclosure may be combined in any suitable manner in one or more embodiments. Those skilled in the art will recognize that the invention can be practiced without one or more of the specific details, or with other methods, components, materials, etc. In other instances, well-known structures, materials, or operations are not shown in detail to avoid obscuring aspects of the invention.

Furthermore, the described features, operations, or characteristics may be combined in any suitable manner in one or more embodiments. It will also be readily understood that the order of the steps or actions of the methods described in connection with the embodiments disclosed may be changed as would be apparent to those skilled in the art. Thus, any order in the drawing or Detailed Description is for illustrative purposes only and is not meant to imply a required order, unless specified to require an order.

Embodiments may include various steps, which may be embodied in machine-executable instructions to be executed by a general-purpose or special-purpose computer (or other electronic device). Alternatively, the steps may be performed by hardware components that include specific logic for performing the steps or by a combination of hardware, software, and/or firmware.

Embodiments may also be provided as a computer program product including a machine-readable storage medium having stored thereon instructions that may be used to program a computer (or other electronic device) to perform processes described herein. The machine-readable storage medium may include, but is not limited to: hard drives, floppy diskettes, optical disks, CD-ROMs, DVD-ROMs, ROMs, RAMs, EPROMs, EEPROMs, magnetic or optical cards, solid-state memory devices, or other types of media/machine-readable medium suitable for storing electronic instructions.

Suitable networks for configuration and/or use as described herein include one or more local area networks, wide area networks, metropolitan area networks, and/or “Internet” or IP networks, such as the World Wide Web, a private Internet, a secure Internet, a value-added network, a virtual private network, an extranet, an intranet, or even standalone machines which communicate with other machines by physical transport of media (a so-called “sneakernet”). In particular, a suitable network may be formed from parts or entireties of two or more other networks, including networks using disparate hardware and network communication technologies.

One suitable network includes a server and several clients; other suitable networks may contain other combinations of servers, clients, and/or peer-to-peer nodes, and a given computer may function both as a client and as a server. Each network includes at least two computers, such as the server and/or clients. A computer may be a workstation, laptop computer, disconnectable mobile computer, server, mainframe, cluster, so-called “network computer” or “thin client”, personal digital assistant or other hand-held computing device, “smart” consumer electronics device or appliance, or a combination thereof.

The network may include communications or networking software, such as the software available from Novell, Microsoft, Artisoft, and other vendors, and may operate using TCP/IP, SPX, IPX, and other protocols over twisted pair, coaxial, or optical fiber cables, telephone lines, satellites, microwave relays, modulated AC power lines, physical media transfer, and/or other data transmission “wires” known to those of skill in the art. The network may encompass smaller networks and/or be connectable to other networks through a gateway or similar mechanism.

Each computer includes at least a processor and a memory; computers may also include various input devices and/or output devices. The processor may include a general purpose device, such as a 80.×.86, Pentium (mark of Intel), 680.times.0, or other “off-the-shelf” microprocessor. The processor may include a special purpose processing device, such as an ASIC, PAL, PLA, PLD, Field Programmable Gate Array, or other customized or programmable device. The memory may include static RAM, dynamic RAM, flash memory, ROM, CD-ROM, disk, tape, magnetic, optical, or other computer storage medium. The input device(s) may include a keyboard, mouse, touch screen, light pen, tablet, microphone, sensor, or other hardware with accompanying firmware and/or software. The output device(s) may include a monitor or other display, printer, speech or text synthesizer, switch, signal line, or other hardware with accompanying firmware and/or software.

The computers may be capable of using a floppy drive, tape drive, optical drive, magneto-optical drive, or other means to read a storage medium. A suitable storage medium includes a magnetic, optical, or other computer-readable storage device having a specific physical configuration. Suitable storage devices include floppy disks, hard disks, tape, CD-ROMs, DVDs, PROMs, random access memory, flash memory, and other computer system storage devices. The physical configuration represents data and instructions which cause the computer system to operate in a specific and predefined manner as described herein.

Suitable software to assist in implementing the invention is readily provided by those of skill in the pertinent art(s) using the teachings presented here and programming languages and tools, such as Java, Pascal, C++, C, database languages, APIs, SDKs, assembly, firmware, microcode, and/or other languages and tools. Suitable signal formats may be embodied in analog or digital form, with or without error detection and/or correction bits, packet headers, network addresses in a specific format, and/or other supporting data readily provided by those of skill in the pertinent art(s).

Several aspects of the embodiments described will be illustrated as software modules or components. As used herein, a software module or component may include any type of computer instruction or computer executable code located within a memory device. A software module may, for instance, comprise one or more physical or logical blocks of computer instructions, which may be organized as a routine, program, object, component, data structure, or the like that performs one or more tasks or implements particular abstract data types.

In certain embodiments, a particular software module may comprise disparate instructions stored in different locations of a memory device, which together implement the described functionality of the module. Indeed, a module may comprise a single instruction or many instructions and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

Much of the infrastructure that can be used according to the present invention is already available, such as: general purpose computers; computer programming tools and techniques; computer networks and networking technologies; digital storage media; authentication, access control, and other security tools and techniques provided by public keys, encryption, firewalls, and/or other means.

We claim:

1. A computer system to allow a pharmacy to serve patients of a plurality of qualified entities, the system comprising:
a processor;
an output device in electrical communication with the processor; and
a memory in electrical communication with the processor, the memory comprising,
an operating system, and
a qualified program management module to perform the method of,
enabling dispensing of drugs to the patients of the plurality of qualified entities from a common inventory of the pharmacy, wherein each of the qualified entities is entitled to reduced cost drugs under a respective qualified program,
monitoring the drugs dispensed to the patients of each of the plurality of qualified entities from the common inventory,
generating a report of the drugs dispensed to patients of a particular qualified entity, wherein the report is generated according to regulations of a respective qualified program of the particular qualified entity, and
enabling the replenishment of the drugs dispensed to the patients of the particular qualified entity, wherein the drugs are replenished according to the regulations of the respective qualified program of the particular qualified entity.

2. The system of claim 1, wherein the regulations of the qualified program of the particular qualified entity provide for bill-to/ship-to replenishment.

3. The system of claim 2, wherein replenishment drugs to replenish the drugs dispensed to the patients of the particular qualified entity are shipped directly to the pharmacy from a manufacturer and/or a wholesaler of the replenishment drugs.

4. The system of claim 3, wherein a payment for the replenishment drugs is made directly by the particular qualified entity to the manufacturer and/or wholesaler of the replenishment drugs.

5. The system of claim 4, wherein the method performed by the qualified program management module further comprises:
enabling reimbursement of the particular qualified entity for a difference between a disbursement cost of the drugs dispensed to the patients of the qualified entity and a cost of the replenishment drugs.

6. The system of claim 1, wherein the method performed by the qualified program management module further comprises:
identifying an alternative, equivalent drug available to replenish the drugs dispensed to the patients of the particular qualified entity.

7. The system of claim 6, wherein identifying the alternative, equivalent drug comprises identifying a reduced cost drug available for purchase by the particular qualified entity.

8. The system of claim 7, wherein the alternative, equivalent drug is a drug available for purchase according to the qualified program of the particular qualified entity.

9. The system of claim 6, wherein the qualified program management module uses a national drug code (NDC) of one or more of the drugs dispensed to the patients of the particular qualified entity to identify the alternative, equivalent drug.

10. The system of claim 1, further comprising an adjudication system, wherein the method performed by the qualified program management module further comprises:
using the adjudication system to monitor drugs dispensed to the patients of the plurality of qualified entities.

11. The system of claim 10, wherein using the adjudication system to monitor drugs dispensed to the patients of the plurality of qualified entities comprises storing data relating to the drugs dispensed to the patients of the plurality of qualified entities in a data structure of the adjudication system.

12. The system of claim 11, wherein the adjudication system data structure complies with a data structure specification promulgated by the National Council for Prescription Drug Programs (NCPDP).

13. The system of claim 11, wherein the adjudication system comprises a coordination of benefits (COB) system, and wherein using the adjudication system to monitor the drugs dispensed to the patients of the plurality of qualified entities comprises storing data relating to the drugs dispensed to the patients of the plurality of qualified entities in a data structure of the COB system.

14. The system of claim 11, wherein the method performed by the qualified program management module further comprises:
filtering one or more adjudication system data structures to obtain data relating to the drugs dispensed to the patients of the plurality of qualified entities.

15. The system of claim 1, wherein a qualified program of one of the plurality of qualified entities is selected from the group consisting of a 340B program, a Group Purchasing Organization (GPO), and a Patient Assistance Program (PAP).

16. A computer readable storage medium, having stored thereon computer readable instruction code for performing a method to allow a contract pharmacy to serve patients of a plurality of qualified entities, the method comprising:
enabling dispensing of drugs to the patients of the plurality of qualified entities from a common inventory of the contract pharmacy, wherein each of the qualified entities is entitled to reduced cost drugs under a respective qualified program;
monitoring the drugs dispensed to the patients of each of the plurality of qualified entities from the common inventory;
generating a report of the drugs dispensed to patients of a particular qualified entity, wherein the report is generated according to regulations of a respective qualified program of the particular qualified entity; and
enabling replenishment of the drugs dispensed to the patients of the particular qualified entity, wherein the drugs are replenished according to the regulations of the respective qualified program of the particular qualified entity.

17. The computer readable storage medium of claim 16, wherein the regulations of the qualified program of the particular qualified entity provide for bill-to/ship-to replenishment.

18. The computer readable storage medium of claim 17, wherein the method further comprises:
enabling the particular qualified entity to provide payment for the replenishment drugs directly to a provider of the replenishment drug.

19. The computer readable storage medium of claim 17, wherein the method further comprises:
enabling reimbursement of the particular qualified entity for a difference between a cost of the drugs dispensed to the patients of the particular entity and a cost of the replenishment drugs.

20. The computer readable storage medium of claim 16, wherein the method further comprises:
identifying an alternative, equivalent drug available to replenish the drugs dispensed to the patients of the particular qualified entity.

21. The computer readable storage medium of claim 20, wherein identifying the alternative, equivalent drug comprises identifying a reduced cost drug available for purchase by the particular qualified entity.

22. The computer readable storage medium of claim 21, wherein the alternative, equivalent drug is available for purchase according to the qualified program of the particular qualified entity.

23. The computer readable storage medium of claim 20, wherein a national drug code (NDC) of one or more of the drugs dispensed to the patients of the particular qualified entity is used to identify the alternative, equivalent drug.

24. The computer readable storage medium of claim 16, wherein the method further comprises:
using an adjudication system of the contract pharmacy to monitor the drugs dispensed to the patients of the plurality of qualified entities.

25. The computer readable storage medium of claim 24, wherein using the adjudication system to monitor the drugs dispensed to the patients of the plurality of qualified entities comprises storing data relating to the drugs dispensed to the patients of the plurality of qualified entities in a data structure of the adjudication system.

26. The computer readable storage medium of claim 25, wherein the adjudication system data structure complies with a data structure specification promulgated by the National Council for Prescription Drug Programs (NCPDP).

27. The computer readable storage medium of claim 25, wherein the adjudication system of the contract pharmacy comprises a coordination of benefits (COB) system, and wherein using the adjudication system to monitor drugs dispensed to the patients of the plurality of qualified entities comprises storing data relating to the drugs dispensed to the patients of the plurality of qualified entities in a data structure of the COB system.

28. The computer readable storage medium of claim 25, wherein the method further comprises:
filtering one or more adjudication system data structures to obtain data relating to drug dispensed to the patients of the plurality of qualified entities.

29. The computer readable storage medium of claim 16, wherein a qualified program of one of the plurality of qualified entities is selected from the group consisting of a 340B program, a Group Purchasing Organization (GPO), and a Patient Assistance Program (PAP).

30. A method to allow a pharmacy to serve patients of a plurality of qualified entities, comprising:
providing for dispensing of drugs to the patients of the plurality of qualified entities from a common inventory of the pharmacy, wherein each of the qualified entities is entitled to reduced cost drug under a respective qualified program;
monitoring the drugs dispensed to the patients of the plurality of qualified entities using a computer system;
automatically generating a report of the drugs dispensed to patients of a particular qualified entity, wherein the report is generated according to regulations of a qualified program of the particular qualified entity;
providing for automatic replenishment of the drugs dispensed to the patients of the particular qualified entity, wherein the replenishment drugs are obtained according to the regulations of the qualified program of the particular qualified entity.

31. The method of claim 30, wherein the pharmacy comprises an adjudication system, the method further comprising:
storing data related to the drugs dispensed to the patients of the plurality of qualified entities in a data structure of the adjudication system.

32. The method of claim 31, wherein generating the report for the particular qualified entity comprises filtering a plurality of adjudication system data structures to obtain data relating to the drugs dispensed to the patients of the particular qualified entity.

33. The system of claim 31, wherein the adjudication system comprises a coordination of benefits (COB) system, and wherein storing data related to the drugs dispensed to the patients of the plurality of qualified entities comprises storing data relating to the drugs dispensed to the patients of the plurality of qualified entities in a data structure of the COB system.

34. The method of claim 31, wherein automatically replenishing the drugs dispensed to the patients of the particular qualified entity comprises filtering a plurality of adjudication system data structures to obtain data relating to the drugs dispensed to the patients of the particular qualified entity.

35. The method of claim 30, further comprising automatically identifying an alternative, equivalent drug to replenish the drug dispensed to the patients of the particular qualified entity using a National Drug Code of one or more of the drugs dispensed to the patients of the particular qualified entity.
